(19) United States
(12) Patent Application Publication
(10) Pub. No.: US 2019/0041333 A1
(43) Pub. Date: Feb. 7, 2019

(54) IMAGING METHOD USING FLUORESENCE AND ASSOCIATED IMAGE RECORDING APPARATUS

(71) Applicant: Schölly Fiberoptic GmbH, Denzlingen (DE)

(72) Inventors: Ingo Doser, Villingen-Schwenningen (DE); Andreas Hille, Villingen-Schwenningen (DE)

(73) Assignee: Schölly Fiberoptic GmbH, Denzlingen (DE)

(21) Appl. No.: 16/050,387

(22) Filed: Jul. 31, 2018

(30) Foreign Application Priority Data
Aug. 1, 2017 (DE) 102017117428.1

(51) Int. Cl. G01N 21/64; A61B 1/04; A61B 1/06; G01J 3/44; A61B 5/00

(52) U.S. Cl. CPC G01N 21/6456; A61B 1/043; G01N 2021/6439; G01J 3/4406; A61B 5/0071; A61B 1/0638

(57) ABSTRACT

For an imaging method for presenting fluorophores (5) by optical excitation, spontaneous emission of fluorescence and detection of same, a single, conventional sensor (6) is used having at least two color channels, which detect an excitation light used to excite the fluorophore (5) and the fluorescence emitted by the fluorophore (5) with different sensitivities. Due to the different spectral distribution of the sensitivity of the color channels, it is possible to separate the component of the excitation light from the component of the fluorescence, in particular in a specific pixel, from one another by processing output signals of said color channels, in particular by conversion into a color space and/or by calculation of color saturation values. From this, the intensity of the fluorescence can be deduced, preferably taking account of a relative luminance measured by the color channels, even though reflected excitation light reaches the color channels, in particular in an unfiltered manner.

### INCORPORATION BY REFERENCE

The following documents are incorporated herein by reference as if fully set forth: German Patent Application No. 10 2017 117 428.1, filed Aug. 1, 2017.

### BACKGROUND

The invention relates to an imaging method, wherein a fluorophore is irradiated with excitation light by a light source emitting in a first spectral range and a fluorescence emitted by the fluorophore in a second spectral range is detected by a sensor. Further, the invention relates to an associated image recording apparatus.

Methods as described at the outset are known per se and used, for example, in fluorescence microscopy or in medical examinations. These methods are based on the physical effect of fluorescence, in which fluorescent dyes, so-called fluorophores or else fluorochromes, are excited with excitation light at an absorption wavelength and, as a result thereof, fluorescence is spontaneously emitted a few nanoseconds later at an emission wavelength; here, as a rule, the spontaneous emission of the fluorescence has lower energy than the excitation light that was previously absorbed by the fluorophore. Therefore, as a rule, the emission wavelength of a fluorophore is also longer than the absorption wavelength that the fluorophore previously absorbed.

In such methods, special optical filters typically ensure that only the light emitted by the fluorophores (fluorescence) is observed. Consequently, the filters prevent the excitation light that was reflected by an object to be observed from disturbing the observation of the fluorescence. This is of particular relevance if the Stokes displacement, i.e., the displacement of the absorption wavelength to the emission wavelength of the fluorophore, is small. Such a separation of the fluorescence from the excitation light is also referred to as “color separation”.

Particularly in the case of medical examinations, fluorophores are introduced into the bloodstream of the patient in order to be able to present blood vessels in detail during the examination. Here, there is often the desire or the specific requirement to be able to also observe the object, for example the tissue surface of an organ, in broadband illumination light, in particular white illumination light, in addition to the observation of the fluorescence. Expressed differently, there consequently is the need for a method with which it is possible to observe both conventional images and images produced by fluorescence, preferably simultaneously and live, with little technical outlay.

The prior art has disclosed imaging methods that use a plurality of sensors with different characteristics for the separate detection of illumination light and fluorescence. By way of example, 3-chip image sensors are already used to this end, said 3-chip image sensors using abruptly responding dichroic filters, i.e., interference filters, for separation purposes. To this end, one of the sensors is configured, for example, to selectively detect the fluorescence with the aid of a dichroic filter while this sensor does not detect the illumination light, or only detects the latter very weakly; conversely, a further image sensor can be configured to detect the illumination light, with a further filter blocking the fluorescence that interferes in the process. However, the technical outlay connected to the application of such methods and apparatus is high; the procurement costs, in particular, for suitable image recording apparatuses are high.

### SUMMARY

The invention is therefore based on the object of providing an imaging method that is improved in comparison with the prior art. In particular, the technical and financial outlay for imaging when using fluorophores should be reduced. Therefore, the invention wants to avoid, in particular, the technical outlay that arises when using a plurality of sensors.

According to the invention, an imaging method with one or more features of the invention is provided for the purposes of achieving this object. Therefore, in particular, in order to achieve the object in an imaging method of the type set forth at the outset, the suggestion is for the sensor to have at least two color channels, the sensitivities of which are distributed differently in the first spectral range and in the second spectral range, and for the at least two color channels in each case to detect the excitation light in the first spectral range and the fluorescence in the second spectral range.

Here, the two spectral ranges also can overlap. In particular, it is consequently possible for a first emission spectrum of the light source, which the light source emits within the first spectral range, to overlap with a second emission spectrum, which the fluorophore emits within the second spectral range. The first/second spectral range consequently can be defined, in particular, by the first/second emission spectrum.

According to the invention, a separation of the fluorescence from the excitation light still is possible, even in the case of a complete overlap of one of the two spectral ranges with the respective other, for as long as the spectral distribution of the excitation light sufficiently differs for separation purposes from that of the fluorescence.

Here, in particular, sensitivity can be understood to be the change in a signal value that is output by a color channel of the sensor in relation to the light intensity incident on the color channel that causes said change.

Since each of the color channels detects both the excitation light and the fluorescence, the output signal of each of the at least two color channels of the sensor depends on an intensity of the excitation light incident on the sensor and an incident intensity of the fluorescence.

A substantial advantage of the method as claimed in claim 1 consists of being able to undertake a separation of the reflected excitation light from the fluorescence without having to resort to specifically matched optical filters in the process. Consequently, the imaging method according to the invention renders it possible, in particular by using a (single) conventional sensor, to easily detect fluorescence and excitation light and distinguish these from one another. This is because, according to the invention, such a separation can already be achieved by signal processing.

Below, this separation concept should initially be explained demonstratively using the example of a CMOS image sensor, which has three color channels (R, G, B), which are each configured as individual color pixels. By way of example, in the case of conventional image sensors with color grids in the form of a Bayer pattern (see U.S. Pat. No. 3,971,065), it is usual for each resolvable pixel, which may be a green pixel, a red pixel or a blue pixel, to be assigned a red-green-blue triple after carrying out so-called “debayering” (or else “demosaicing”). Since each pixel can only receive the value of one color channel, the color information is incomplete in this case. Accordingly, the missing color information in a pixel must be established by interpolation (see U.S. Pat. No. 4,642,678).

Moreover, the prior art has disclosed further embodiments of such color grids, which differ in respect of the number of color filters/color pixels and their respective arrangement. However, a person skilled in the art can readily also apply the description below to other sensors and color grids, in particular those with two, or more than three, color channels, without loss of generality. However, in this case, it is particularly advantageous for the separation method explained below for the employed sensor to have different spectral filters, in particular in the form of pixels, which are arranged in such a way that an entire spectral range to be detected can be captured by spatially adjacent spectral filters or pixels. This is because this can obtain a high spatial resolution of the spectral imaging. Consequently, the method explained below can be applied, for example, to color information of individual pixels of a sensor, which were established by means of debayering (see above).

With reference to the CMOS image sensor introduced in the previous paragraph, the following applies to each of its pixels:

$$S = ( \begin{matrix}R \\ G \\ B\end{matrix} ) = ( \begin{matrix}{R}_{A} \\ {G}_{A} \\ {B}_{A}\end{matrix} ) + ( \begin{matrix}{R}_{F} \\ {G}_{F} \\ {B}_{F}\end{matrix} ) \qquad ( 1 )$$

Here, R, G, B are signal values output by the individual color channels of the sensor, which correlate to an overall intensity I, and the spectral distribution thereof, incident on the color channels. As mentioned previously, individual color information items (R/G/B) could have been established by interpolation in this case.

The vector S presents itself as the sum of contributions of the excitation light reflected by the observed surface (index A) and of the fluorescence emitted by the fluorophore (index F). This is because if both excitation light and fluorescence strike an individual R/G/B pixel of the sensor, both light components contribute to the creation of the signal value of the respective color channel.

For the excitation light, the reflection properties of the illuminated surface can be taken into account, at least approximately, as

$$( \begin{matrix}R \\ G \\ B\end{matrix} ) = {V}_{A} ( \begin{matrix}{r}_{A} \\ {g}_{A} \\ {b}_{A}\end{matrix} ) + ( \begin{matrix}{R}_{F} \\ {G}_{F} \\ {B}_{F}\end{matrix} ) \qquad ( 2 )$$

Here, the components r_{A}, g_{A}, b_{A} should be understood, in each case, as a product of the emission spectrum of the exciting light source, the reflection spectrum of the illuminating surface and the sensitivity of the respective color channel (R/G/B), while V_{A} should be understood to be a scaling factor that allows different intensities of the excitation light to be modeled. A less precise approximation that, however, is expedient in certain situations lies in only considering the emission spectrum of the light source and the sensitivities of the color channels and neglecting the reflection properties of the surface to be observed. This approximation lends itself when using narrowband excitation light in particular since the influence of the reflection spectrum of the surface can be neglected in this case.

For a given fluorophore, the assumption can be made that the latter emits a spectrum that is constant. Hence, the preceding equation (2) can be differentiated further:

$$S = ( \begin{matrix}R \\ G \\ B\end{matrix} ) = {V}_{A} ( \begin{matrix}{r}_{A} \\ {g}_{A} \\ {b}_{A}\end{matrix} ) + {V}_{F} ( \begin{matrix}{r}_{F} \\ {g}_{F} \\ {b}_{F}\end{matrix} ) = {V}_{A} A + {V}_{F} F \qquad ( 3 )$$

Here, r_{F}, g_{F}, b_{F} should be understood, in each case, as a product of the emission spectrum of the fluorophore and the sensitivity of the respective color channel (R/G/B), while V_{F} should be understood to be a scaling factor that allows different intensities of the fluorescence to be modeled.

The separation of the contributions of the excitation light V_{A} and of the fluorescence V_{F} at a pixel therefore presents itself approximately as the solution of the linear combination of equation (3), where the vectors A and F are determined by the choice of the sensor, the fluorophore and the exciting light source.

It is understood that equation (3) is solvable precisely when the two vectors A and F are linearly independent. By way of example, the solution is provided by virtue of the vector S being projected along A onto F and along F onto A. The invention has now recognized that linear independence is provided, for example, if the sensor has at least two color channels, the wavelength-dependent sensitivities of which are distributed differently in the spectral range of the excitation light and in the spectral range of the fluorescence.

Here, in order to resolve equation (3) and consequently be able to separate the components of the excitation light and of the fluorescence from one another, measurement signals of at least two independent color channels of the sensor must be present. Particularly when using an image sensor, it consequently becomes possible to distinguish an intensity of the fluorescence, preferably in a spatially resolved manner, from an intensity of the excitation light by means of the sensor.

Alternatively, or in a complementary manner, the features of the second coordinate claim directed to an imaging method are provided for achieving the aforementioned object. In particular, an alternative proposition, or a proposition complementing the approach described above, according to the invention in an imaging method of the type set forth at the outset for the purposes of achieving the aforementioned object is that, in each case, the excitation light and the fluorescence produce signals in at least two color channels of the sensor, i.e., in particular, in R/G/B pixels of the sensor, which each can be assigned to different hues and/or different color saturations. To this end, there can be an appropriate selection of the sensor, the light source and the fluorophore. What is advantageous here is that there can be a separation of the fluorescence from the excitation light using the differences in the hues produced by the excitation light and by the fluorescence and/or in the produced color saturations, in particular using unit vectors yet to be explained in more detail.

Reference is made here to the fact that hues and color saturations can be understood to mean, in particular, specific properties of color information that are obtained from signals of the color channels of the sensor. By way of example, conventional image sensors output an associated RGB triplet when a pixel is irradiated with a saturated color (and used as intended), in which RGB triplet only one of the three colors R/G/B has a high amplitude. By contrast, different hues emerge by rotating the vector that is composed of the color information R, G, B of a triplet. In certain applications of the method according to the invention, for example when detecting infrared light (not visible to humans), hues or color saturations within the meaning of the invention can therefore precisely no longer be related to hues and color saturations as perceived by the human eye.

According to the invention, the object can also be achieved by further advantageous embodiments as described below and in the claims.

By way of example, using the approaches presented above, it is possible, in particular, to produce image signals that correspond to an intensity distribution of the fluorescence or of the excitation light. According to a preferred configuration, the “color separation” required to this end, as described above, can be realized particularly easily if the sensor has a color saturation upon irradiation with excitation light that differs from a color saturation that the sensor has upon irradiation with the fluorescence.

In general, the term color saturation describes how strongly a colored stimulus differs from an achromatic stimulus, independently of the brightness thereof. Thus, saturated colors are distinguished by a high spectral purity and high color intensity. In relation to the imaging method according to the invention discussed here, color saturation can be understood to mean, in particular, a color saturation value that correlates with the equality or inequality of the sensitivities of the color channels. By way of example, if two or three color channels exhibit an approximately equal sensitivity at a specific wavelength, the sensor can have or output a correspondingly low color saturation for this wavelength. Conversely, as a rule, the sensor can have or output a high color saturation for a specific wavelength particularly when the sensitivities of its colors channels differ (in particular differ strongly) for this wavelength.

In addition to brightness and color saturation, the hue represents the third basic property of a color. The hue, which represents one of the three possible coordinates in the HSV color space, describes, inter alia, the color perception, on the basis of which red, green or blue colors, for example, are distinguished by us. The invention has now recognized that different spectral components of a light spectrum which consists of a superposition of two emission spectra and which is recorded by a sensor can be distinguished on the basis of different hues. This applies, in particular, if the two emission spectra produce approximately the same color saturations on the sensor. Consequently, a “color separation” can easily be realized according to a further configuration if the exciting illumination and the fluorophore are chosen precisely in such a way that the excitation light and the fluorescence have hues that are distinguishable from one another by the at least two color channels of the sensor. This distinguishability may even still be ensured when the sensor has substantially the same color saturations within the first and the second spectral range. Here, a hue can be understood to mean in particular a distribution of signals, specific for a certain light spectrum, which is output by the at least two color channels of the sensor.

Thus, the first and the second spectral range can be chosen precisely in such a way that light from the first spectral range is distinguishable on the basis of measured hues from the light from the second spectral range with the aid of the color channels of the sensor. In particular, this is also possible when the first and the second spectral range produce approximately the same color saturations on the sensor.

Therefore, a color saturation value can be calculated, for example, from output values or signals of the color channels of the sensor at a specific wavelength. In particular, this wavelength can be an emission wavelength of the fluorophore or of an exciting light source. By way of example, a color saturation can be established as a quotient of signals of two color channels. When using an RGB sensor in particular, a color saturation can be calculated in a manner known per se by a conversion from the RGB color space into the HSV (hue-saturation-value) color space.

By way of example, a color saturation value that is higher as differences in the light intensity detected by the individual color channels increase can be formed from output values of the color channels of the sensor, i.e., for example, of red, green and blue sensor elements of an RGB sensor. If red, green and blue sensor elements were to output approximately the same light intensities in such a case, a correspondingly low color saturation value would be formed following the method. Conversely, the spectral components of the incident light detected by the color channels of the sensor would have significantly different strengths in this case should the color saturation be high.

However, some image sensors, such as RGB sensors, for example, have the property that is useful for the invention but unremarkable or even unwanted during normal application that the sensitivities for the individual color channels deviate strongly from one another in a first spectral range while they are virtually at the same level in a second spectral range. Now, this property is exploited by the invention in such a way that what is achieved by suitable choice of the light source and of the fluorophore is that, for example, the excitation light lies in the first spectral range and the fluorescence lies in the second spectral range. Hence, the excitation light and the fluorescence lie precisely in those spectral ranges that can be easily separated from one another on account of the wavelength-dependent sensitivities of the color channels.

Accordingly, it is expedient for a particular robust separation, according to an advantageous configuration, if the light source, the fluorophore and the sensor are chosen in such a way that the excitation light produces a high color saturation on the sensor. In this case, it is preferable if the fluorescence produces a lower color saturation on the sensor in comparison therewith. In such a case, it is possible to assign light components with a low color saturation that are detected by the image sensor to the fluorescence, while light components with a high color saturation are assignable to the excitation light.

However, alternatively, a robustness of the separation is also given if the excitation light produces a low color saturation on the sensor. In this case, it is preferable if the fluorescence produces a higher color saturation on the sensor in comparison therewith. In such a case, it is possible to assign image components with a low color saturation that are detected by the image sensor to the excitation light, while light components with a high color saturation are assignable to the fluorescence.

When applying a method according to the invention, it is possible in particular in certain configurations to dispense with optical pre-filters for suppressing the excitation light or fluorescence. Consequently, provision can be made according to a further configuration of the method for light from the first spectral range and/or the fluorescence from the second spectral range and/or light from a further spectral range to reach the sensor unfiltered. The light from a further spectral range, depending on application, can be a spectrum of reflected excitation light, for example, or a spectrum of an additional illumination source, for example. What is advantageous here is that a further spectrum in addition to the fluorescence can be used for imaging purposes. Consequently, one and the same hardware can also be used, in particular, for applications with broadband spectral ranges or spectral ranges deviating from the fluorescence without the restrictions that occur during the conventional use of optical filters.

As described above and unlike conventional methods, the invention facilitates the separation of fluorescence and excitation light from one another by processing signals of the color channels of the sensor. Therefore, an advantageous configuration provides for an intensity of the fluorescence to be separated from an intensity of the excitation light by processing signals from the at least two color channels. Here, digital signal processing should be considered advantageous since it is implementable in a particularly simple manner using available hardware. Further, it is advantageous in this configuration if the separation of the two intensities is brought about in a spatially resolved manner. This is because this renders it possible to generate complex fluorescence images. Additionally, provision can be made, in particular, for the separation to be performed using color saturation values or hues that are obtained from the signals of the color channels, in particular by conversion into a color space. At this point, too, reference is made, once again, to the fact that the terms of hue and color saturation in certain applications of the method according to the invention need not necessarily correspond to human perception. Accordingly, color spaces not accessible to human perception can also be used for the separation according to the invention.

In order to improve the applicability of the method, a further configuration proposes that an automated algorithm is used to separate the fluorescence from the excitation light. By way of example, the algorithm can be implemented in an evaluation circuit of the image sensor or a downstream camera controller. Here, it is preferable if the algorithm has an adjustable configuration. It is particularly preferred if the algorithm is adjustable by a user, preferably during the application of the method, to different fluorophores and/or exciting light sources. This is because this allows the method to be adapted flexibly and quickly to different applications.

According to a further advantageous configuration of the method, a particularly simple separation can be achieved by virtue of signals of the color channels being converted into a color space that has a saturation value as a coordinate or a degree of freedom. Here, in particular, provision can be made for color saturation values that are obtained from the signals by the conversion to be assigned, preferably with the aid of a table, to corresponding components of the fluorescence or of the excitation light. Using this approach, it is possible, in particular, to produce image signals, which correspond to an intensity distribution of the fluorescence or of the excitation light. Here, using a relative luminance recorded by the sensor for calculating the intensities is proposed. By way of example, an HSV (hue, saturation, value) color space or an HSL (hue, saturation, lightness) color space or an HSI (hue, saturation, intensity) color space can be used as such a color space. Here, the conversion of RGB to HSV, HSL or HSI is known per se.

As an alternative or in addition to the approach described above of a conversion from an RGB color space into an HSV color space for the purposes of establishing color saturation values, provision according to a further configuration can be made for a color vector in each case to be stored as a unit vector for an overall intensity detected by the color channels and/or for the light source and/or for the fluorophore. For an RGB sensor, these color vectors correspond to the vectors S, A and F from equation (3) explained above. With the aid of such color vectors, it is possible, for example, to set up a linear system of equations from which the components of the fluorescence and/or of the excitation light can be calculated. If a relative luminance measured by the sensor is additionally taken into account, it is possible to establish the intensities of the fluorescence and/or of the excitation light.

According to an advantageous configuration, the component of the fluorescence can be established, in particular, by computational projection of the intensity vector S detected (by the color channels) along the color vector A of the (exciting) light source onto the color vector F of the fluorophore. In an analogous fashion, the component of the excitation light can be established, in particular, by computational projection of the detected intensity vector S along the color vector F of the fluorophore onto the color vector A of the light source.

These separation methods based on color vectors already can be carried out in the presence of at least two color channels of a sensor. Here, according to the invention, a robust separation can be obtained precisely when the color vectors stored for the excitation light and the fluorescence are linearly independent. This can be achieved by suitably matching the filter characteristics of the color channels of the sensor to the employed light source and the employed fluorophore, wherein, in the case of a sensor being present, it is naturally also possible to choose the light source and the fluorophore accordingly. What is decisive here is that an overall intensity detected by the color channels of the sensor can be established as a sum of two vectors, wherein the two vectors describe components of the signals output by the color channels of the sensor that are caused by the fluorescence and by the excitation light (in this respect, see equation (1) above).

The imaging methods presented here are advantageous, in particular, to endoscopic examinations as only a single sensor and correspondingly little installation space have to be used in order to be able to detect both excitation or illumination light and fluorescence that is emitted by a fluorophore. Hence, the invention opens up new imaging options, particularly for endoscopic applications. According to a preferred embodiment of the invention, a conventional image sensor, in particular a CMOS sensor and/or Bayer sensor, can be used accordingly as a multi-channel sensor. Expressed differently, different sensor elements, in particular individual pixels, of an image sensor with respectively assigned color filters thus can be used as color channels according to the invention. Thus, in particular, provision can be made for the color channels to have subtractive filters for separating spectral light components.

For a robust separation, it is moreover advantageous if the sensor has at least three color channels. By way of example, the sensor can have sensor elements for detecting red, green and blue light. Here, it is preferable if these sensor elements also are used to detect the fluorescence. In order to ensure the applicability of the method to fluorophores that fluoresce in the infrared, too, provision can further be made according to a preferred configuration for the aforementioned sensor elements or further sensor elements of the sensor to detect infrared light.

Certain fluorophores, e.g., indocyanine green (ICG), emit in the infrared. Consequently, a method according to the invention can be configured precisely in such a way that the second spectral range of the fluorescence lies partly or completely above a wavelength of 780 nm.

Other fluorophores in turn, e.g., ALA-5, absorb ultraviolet to blue light and emit red light. Consequently, a method according to the invention also can be configured precisely in such a way that the second spectral range of the fluorescence lies partly or completely below a wavelength of 700 nm.

According to a specific configuration, a further improvement of the imaging can be reached by virtue of a narrowband light source being used to excite the fluorophore. By way of example, the width of the emission spectrum of the light source can be less than 50 nm. By choosing a narrowband light source, the fluorophore can be excited and targeted in an efficient manner, on the one hand. On the other hand, the restricted spectral width ensures that the entire excitation light is reflected with virtually the same strength from a surface to be examined such that this reflection can be approximated well. Expressed differently, the reflection spectra of the surfaces illuminated by the excitation light in this case are negligible for the separation. The error arising as a result of this omission sinks in this case with decreasing width of the exciting light spectrum.

Finally, particularly if use is made of a further broadband illumination source, the narrowband excitation light can be efficiently suppressed in the optical path of the sensor with the aid of a notch filter. In this case, the color channels can detect further illumination light in addition to the fluorescence, said further illumination light being able to be separated from the fluorescence in the same way as the excitation light.

What may occur in some applications is that an absorption spectral range of the fluorophore, i.e. a spectral range in which same absorbs light, overlaps with the second spectral range, in which the fluorophore emits fluorescence. In principle, such an overlap is acceptable when applying a method according to the invention. However, to improve the imaging it is preferable, in general and especially in such a case, if an emission wavelength, at which the light source present for excitation purposes exhibits maximum light emission, lies outside of the second spectral range of the fluorescence. This is because this can ensure, in particular, that the excitation light does not swamp the fluorescence.

According to a further configuration, it is moreover considered advantageous if the emission wavelength mentioned above, at which the light source exhibits maximum light emission, is shorter than an absorption wavelength of the fluorophore, at which the latter exhibits maximum light absorption. This is because this prevents the light spectrum used for excitation from overlapping with the fluorescence.

As mentioned at the outset, there is a need to capture fluorescence images and conventional images obtained by illumination light with one and same hardware in the simplest possible manner. In order to achieve this specific partial object, provision is made according to a particularly advantageous configuration of the method for the sensor to detect light in a third spectral range besides the excitation light and the fluorescence. Consequently, in this configuration, the sensor can capture and process light components that originated neither from the exciting light source nor from the fluorophore but instead, for example, from a further illumination light source.

By way of example, if use is made of a sensor which, in addition to conventional RGB pixels, also has pixels that can be used to capture infrared light, it is possible to capture broadband illumination and/or excitation light, and infrared fluorescence.

A further option, which will still need a more detailed description, includes the use of a time sequential illumination. In such a case, it is possible to use both fluorescence and light of a further broadband illumination light source for imaging, even when using conventional RGB sensor. Consequently, it is possible to record conventional images and fluorescence images using only a single sensor. Here, in particular, the sensitivities of the at least two color channels of the sensor can be distributed differently in the third spectral range and in the first or the second spectral range.

As a source for the excitation light, use can be made, in particular, of NIR LEDs or IR lasers or, for example, UV LEDs, too. Therefore, it may be the case that the excitation light has spectral components that lie above or below an absorption spectral range of the fluorophore. Such components can also be detectable by the sensor and therefore can be used for conventional imaging.

According to a further specific configuration, a first image consequently can be obtained with the sensor by detecting the excitation light in the first spectral range or by detecting the fluorescence in the second spectral range, while a second image is obtained with the same sensor by detecting broadband illumination light in a, or the, third spectral range which has already been explained above. Here, for the reasons explained above, it is advantageous if the light source for the excitation light is a narrowband first light source. This is because this then allows a second light source, in particular, to be used to produce the illumination light. Hence, it is possible to obtain both a high excitation efficiency and outstanding conventional imaging.

Should conventional images and fluorescence images be captured by a sensor using one of the above-described methods, provision can be made according to further configuration for detecting the fluorescence or excitation light and detecting the illumination light to be undertaken alternately. To this end, in particular, the two aforementioned light sources can be operated alternately, preferably at half a frequency used for imaging (half the “frame rate”). Hence, a time-sequential application of a method according to the invention is described, by which it is possible to record both fluorescence images and conventional images obtained with broadband illumination, even if use is made of a conventional RGB sensor.

Visualization methods known per se can be used for simultaneously displaying conventional images and images produced by means of fluorescence. By way of example, an overall image can be visualized to a user, in particular in real time, said image being produced by a juxtaposition or superposition of the two image types explained above. Here, the individual images or the overall image also can be subjected to image preparation and/or post-processing in order to improve the representation.

However, even without the use of an additional illumination source, it is already possible to obtain two different images when separating the excitation light from the fluorescence. These images can represent, firstly, a surface illuminated by the excitation light and, secondly, a fluorescence signal produced at the surface. Two such images also can be composed, preferably in real time, to form an overall image that is produced from a juxtaposition or superposition of the two separated images. Here, in particular, a first image signal of the fluorescence light can be visualized as a grayscale value image or in a false color representation and a second image signal of the excitation light can be visualized as a grayscale value image, for example. Using this approach, it is possible to produce overall images with a high information content.

Finally, in order to achieve the object specified at the outset, an image recording apparatus is provided. It has appropriate sensor and a processor for carrying out one of the imaging methods described above. In particular, this image recording apparatus can comprise a data processor that is configured to separate the fluorescence from the excitation light.

According to the invention, it may be necessary to remove the infrared (IR) cutoff filters that are typically used with conventional sensors in the case of fluorescence applications in the infrared range, i.e., when using fluorophores with infrared emission wavelengths. Normally, such a filter is mandatory, particularly in the case of applications that use halogen light or natural daylight for illumination purposes, in order to avoid overexposure of the sensor. Here, the invention has recognized that such a filter may be dispensable, particularly in the case of endoscopic applications, such that a conventional sensor, with omission of an IR cutoff filter, can be used for detecting infrared fluorescence. This is because, firstly, the light of light sources such as LEDs that are used in an endoscopy typically only has small IR components and, secondly, these can be suppressed by filters attached to the light source. Consequently, the image recording apparatus can be configured, in particular, without an optical pre-filter.

Finally, it should be mentioned that the methods discussed here could be used in an advantageous manner, in particular, for applications in neurosurgery, plastic surgery, reconstructive surgery and coronary surgery, for perfusion assessment of organs and tissue, for presenting the gallbladder or for visual assistance when finding and presenting lymph nodes.

### BRIEF DESCRIPTION OF THE DRAWINGS

Now, the invention will be described in more detail on the basis of exemplary embodiments without being restricted to these exemplary embodiments.

Further exemplary embodiment emerge by combining the features of individual claims or of a plurality of claims among themselves and/or with individual features, or a plurality of features, of the respective exemplary embodiment. Consequently, it is possible, in particular, to obtain embodiments of the invention from the following description of a preferred exemplary embodiment in conjunction with the general description, the claims and the drawings.

In the figures:

FIG. 1 shows a schematic view of an image recording apparatus according to the invention,

FIG. 2 shows a diagram for elucidating a specific configuration of the imaging method according to the invention,

FIG. 3 shows a first application example of an imaging method according to the invention using the ICG fluorophore, and

FIG. 4 shows a second application example of an imaging method according to the invention using the ALA-5 fluorophore.

### DETAILED DESCRIPTION

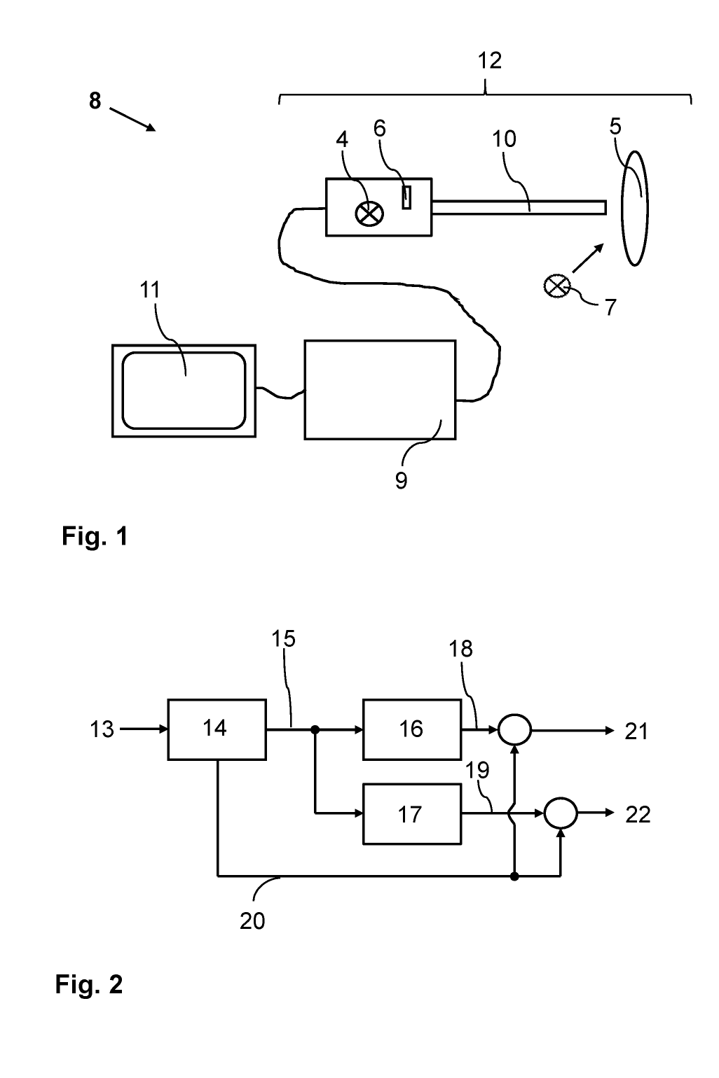

FIG. 1 shows an image recording apparatus according to the invention, denoted as a whole by 8, said image recording apparatus being part of an endoscopic arrangement 12. By an endoscope 10, excitation light A from a first spectral range 1 of a first narrowband light source 4 is steered onto a surface to be examined. Additionally, the surface is illuminated by a second light source 7 with broadband illumination light from a third spectral range 3. On the surface, or therebelow, there is a fluorophore 5, which is irradiated by the excitation light of the first light source 4, which is consequently excited and which subsequently spontaneously emits fluorescence F in a second spectral range 2.

An individual sensor 6 arranged in the endoscope 10, said sensor being configured as a conventional Bayer image sensor with three color channels R, G, B (for red, green, blue) for each pixel, detects the fluorescence, a part of the excitation light from the first light source 4 that was reflected by the surface and a part of the illumination light from the second light source 7 that was reflected by the surface. No pre-filter is used here. Consequently, all these light components reach the sensor surface unfiltered and they are only spectrally decomposed by the subtractive filters of the individual pixels of the color channels R, G, B. This means that the sensor 6 detects infrared components of the fluorescence, in particular.

The color channels R, G, B used to detect light, having subtractive filters of the sensor 6, each delivers output signals in the process, said output signals being processed by a downstream camera controller 9. The camera controller 9 carries out the imaging method according to the invention and, in the process, separates the fluorescence F from the excitation light A. The second light source 7 remains deactivated during this imaging.

The fluorescence F is separated from the excitation light A by an automated algorithm that carries out the above-described computational operations or signal processing. Consequently, it is subsequently possible to present separated images by a monitor 11, said separated images reproducing the illuminated surface and being obtained by detecting the broadband illumination light in a third spectral range. On the other hand, it is possible to display on the monitor 11 a fluorescence image that was obtained by the sensor 6 by a spatially resolved detection of the fluorescence.

In order to simplify the separation of the individual light components, the two light sources 4 and 7 can also be operated alternately, for example with a frequency of 30 Hz. Since spontaneous emission of the fluorophore 5 is effected within nanoseconds and decays correspondingly quickly, only the excitation light has to be separated from the fluorescence in this case when the second light source 7 is deactivated (and the first light source 4 is activated). In the case of such a high changing frequency, it is possible, in particular, to present live images to a user, said live images being composed of a superposition of a fluorescence image and a conventional image that was recorded by the second light source 7.

FIG. 2 explains an imaging method according to the invention, more precisely the method step of “color separation”, when using conventional RGB sensor. An output signal of the RGB sensor is used as input variable 13, said output signal containing signal values of the three color channels R/G/B. This RGB sensor output signal 13 is initially converted into the HSV color space by an HSV conversion 14. Consequently, after conversion, a color saturation value 15, a hue and a relative luminance 20 ascertained from the RGB sensor output signal 13 are available. With the aid of two lookup tables 16 and 17 (stored in a memory accessed by the processor), the respective components 18 and 19 of the fluorescence F and of the excitation light A are established from the color saturation value 15 that is established by conversion. The lookup tables 16 and 17 in this case are based on knowledge of the exciting light spectrum 1, of an approximation of the reflection properties of the observed surface and of the emitted fluorescence spectrum 2. Consequently, after multiplication with the relative luminance 20, it is possible to output the intensity components 21 and 22 of the fluorescence and of the excitation light, respectively, which are detected by the sensor.

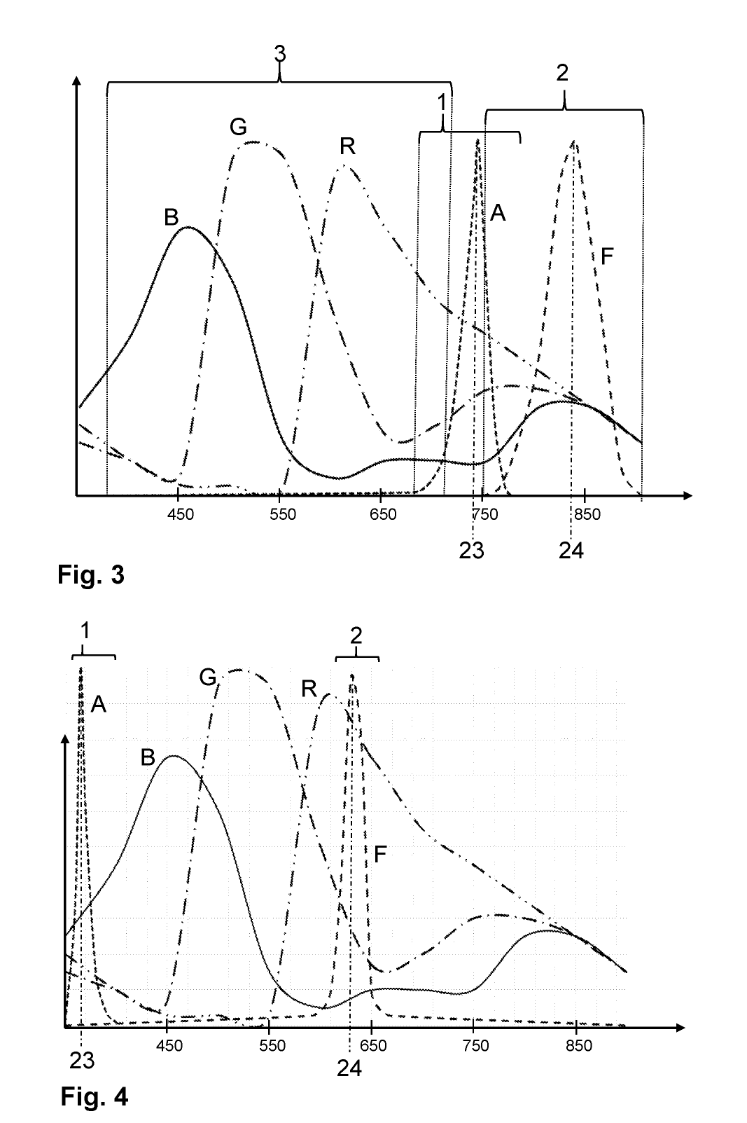

The separation method based on HSV conversion can be understood vividly on the basis of the exemplary embodiment illustrated in FIG. 3: Shown are the wavelength-dependent sensitivities of the three color channels R, G and B of the sensor 6 from FIG. 1, which are denoted by the letters R, G and B in the diagram. The horizontal axis of the diagram specifies the wavelength in nanometers.

As can be easily identified in FIG. 3, the blue color channel B of the RGB sensor 6, for example, exhibits a high sensitivity at a wavelength of approximately 440 nm, whereas the green and the red color channel have an extremely low sensitivity at this wavelength. Conversely, the red color channel R is particularly sensitive at a wavelength of approximately 620 nm, while the green and the blue channel only respond weakly at this wavelength. This characteristic is produced by subtractive color filters arranged on the individual pixels of the RGB sensor 6. For infrared wavelengths, for example, above 780 nm, the three color channels R, G, B by contrast exhibit approximately the same sensitivities, with the three sensitivity curves merging into one another above 850 nm. This specific characteristic is due, on the one hand, to the subtractive color filters having comparable transmission properties for infrared wavelengths and, on the other hand, to the sensitivity of the sensor 6 reducing overall for infrared wavelengths.

FIG. 3 likewise illustrates the emission spectrum 1 of an IR LED, which serves as exciting light source 4. The first spectral range 1 thereof reaches from approximately 680 nm to approximately 760 nm, with a maximum of the emission at an emission wavelength 23 of approximately 740 nm. Further, a second spectral range 2 of the ICG (indocyanine green) fluorophore is shown, which reaches from approximately 750 nm to approximately 950 nm, with a maximum of the emission at an emission wavelength 24 of approximately 840 nm.

By contrast, the absorption spectrum of ICG, which reaches from approximately 600 nm to approximately 900 nm with a maximum of the absorption at a wavelength of approximately 800 nm, is not illustrated.

It is clear from FIG. 3 that the first spectral range 1 of the light source 4 and the second spectral range 2 of the fluorophore 5 overlap slightly in this exemplary embodiment, to be precise in an overlap region at approximately 760 nm.

As already explained on the basis of FIG. 1, the sensor 6 detects both the excitation light and the illumination light. In view of FIG. 3, for the specific exemplary embodiment shown there, this means that the excitation light A and the fluorescence F each produce different R/G/B signal components: while the fluorescence F produces approximately equal R/G/B components and hence low color saturations on the sensor, the red color channel responds more strongly to the excitation light A than the green color channel and much more strongly than the blue color channel, and so a comparatively higher color saturation of the excitation light A is produced. It is understood that different hues can also be assigned to the excitation light A and the fluorescence F, respectively, in an analogous manner by processing the R/G/B components.

With reference to the respective emission wavelengths 23 and 24, the sensor 6 consequently has a color saturation in the first spectral range 1, in particular at the emission wavelength 23, of the excitation light A that differs from a color saturation that the sensor 6 has within the second spectral range 2, in particular at the emission wavelength 24, of the fluorescence.

Producing different color saturation values is substantially simplified by virtue of the central emission wavelength 23 of the excitation light A lying outside of the second spectral range 2 of the fluorescence F. At the same time, the emission wavelength 23 in the exemplary embodiment shown in FIG. 3 lies at approximately 740 nm, as mentioned previously, and hence only approximately 60 nm below the absorption wavelength of approximately 800 nm, at which ICG has a maximum light absorption. Therefore, a particularly efficient excitation can be obtained using the IR LED.

Consequently, in the example shown in FIG. 3, the excitation light A produces a high color saturation on the sensor 6 while the fluorescence F produces a lower color saturation in comparison therewith. Expressed in a simplified manner, this means the following for the example shown in FIG. 3: a pixel that has a high color saturation contains more fluorescence than a pixel that has a low color saturation. Consequently, the component of the fluorescence or the component of the excitation light in the respective pixel can be deduced on the basis of the calculated color saturation after the HSV conversion.

FIG. 3 also indicates a third spectral range 3, within which the second light source 7 from FIG. 1 emits a broadband illumination light (not illustrated). This illumination light likewise can be detected by the sensor 6 and serves to record conventional images of the surface using the sensor 6. To this end, the two light sources can be operated, e.g., in succession or, in a particularly advantageous manner, in alternation. Using the variant mentioned last, it is possible to continuously obtain both conventional images and fluorescence images.

FIG. 4 shows a further application example of an imaging method according to the invention or image recording apparatus according to the invention. Here, use is made of the same RGB sensor as in FIG. 3. The exciting light source is a UV-A-LED, which emits excitation light in a first spectral range 1 with an average emission wavelength of approximately 370 nm. ALA-5 (5-aminolevulinic acid) is used as a fluorophore. ALA-5 absorbs ultraviolet to blue light and emits spontaneous red fluorescence in a second spectral range 2 with an average emission wavelength 24 at approximately 640 nm. Consequently, the first and the second spectral range 1, 2 precisely do not overlap in the example shown in FIG. 4. The reason for this lies in the comparatively large Stokes displacement of ALA-5.

Consequently, in the example shown in FIG. 4, the excitation light A produces a low color saturation on the sensor 6 while the fluorescence F produces a higher color saturation in comparison therewith.

A further possible application of a method according to the invention lies in the presentation of structures using a conventional RGB sensor and fluorescein, a fluorophore that exhibits a spontaneous emission of green light with an emission wavelength 24 of 514 nm. Here, the light used for excitation and/or illumination purposes can lie in a wavelength range in which sensitivities of individual color channels of the sensor are virtually the same, such that correspondingly low color saturation values are produced by the image sensor. By contrast, the fluorescence emitted by fluorescein lies in a wavelength range in which the sensitivities of the individual color channels of conventional image sensors are typically very different, and so, correspondingly, high color saturation values are detected by the sensor.

In conclusion, for an imaging method for presenting a fluorophore 5 by optical excitation, spontaneous emission of fluorescence and detection of same, the suggestion is to use a single, conventional sensor 6 having at least two color channels, which detect an excitation light used to excite the fluorophore 5 and the fluorescence emitted by the fluorophore 5 with different sensitivities. Due to the different spectral distribution of the sensitivity of the color channels, it is possible to separate the component of the excitation light from the component of the fluorescence, in particular in a specific pixel, from one another by processing output signals of said color channels, in particular by conversion into a color space and/or by calculation of color saturation values. From this, the intensity of the fluorescence can be deduced, preferably taking into account a relative luminance measured by the color channels, even though reflected excitation light reaches the color channels, in particular in an unfiltered manner (see FIG. 3).

### LIST OF REFERENCE SIGNS

1 First spectral range
2 Second spectral range
3 Third spectral range
4 (First) light source (excitation light)
5 Fluorophore
6 Sensor
7 Second light source (illumination light)
8 Image recording apparatus
9 Camera controller
10 Endoscope
11 Monitor
12 Endoscopic arrangement
13 RGB sensor output signal
14 HSV conversion
15 Color saturation value
16 Lookup table 1
17 Lookup table 2
18 Component of the fluorescence
19 Component of the excitation light
20 Relative luminance
21 Intensity of the fluorescence
22 Intensity of the excitation light
23 Emission wavelength (of the first light source)
24 Emission wavelength (of the fluorophore)
R Red color channel
G Green color channel
B Blue color channel
A Excitation light
F Fluorescence

1. An imaging method, comprising:
irradiating a fluorophore (5) with excitation light by a light source (4);
detecting light emitted by the fluorophore (5) in a first spectral range (1) and a fluorescence emitted by the fluorophore (5) in a second spectral range (2) using a sensor (6);
wherein the sensor (6) has at least two color channels (R, G, B), sensitivities of said at least two color channels are distributed differently in the first spectral range (1) and in the second spectral range (2), and the at least two color channels (R, G, B) in each case detect the excitation light in the first spectral range (1) and the fluorescence in the second spectral range (2).

2. The imaging method according to claim 1 wherein the excitation light and the fluorescence produce signals in the at least two color channels which are assigned to at least one of different hues or different color saturations.

3. The imaging method as claimed in claim 1, wherein the sensor (6) has a color saturation upon irradiation with the excitation light that differs from a color saturation that the sensor (6) has upon irradiation with the fluorescence.

4. The imaging method as claimed in claim 1, wherein the excitation light and the fluorescence have hues that are distinguishable from one another by the at least two color channels of the sensor (6), and the sensor (6) has substantially the same color saturations within the first and the second spectral range (1, 2).

5. The imaging method as claimed in acclaim 1, wherein the light source (4), the fluorophore (5) and the sensor (6) are chosen such that the excitation light produces a high color saturation on the sensor (6), and the fluorescence produces a lower color saturation on the sensor (6) in comparison therewith, or the excitation light produces a low color saturation on the sensor (6), and the fluorescence produces a higher color saturation on the sensor (6) in comparison therewith.

6. The imaging method as claimed in claim 1, wherein at least one of light from the first spectral range (1), the fluorescence from the second spectral range (2), or light from a further spectral range reaches the sensor (6) unfiltered.

7. The imaging method as claimed in claim 1, further comprising separating an intensity of the fluorescence from an intensity of the excitation light by processing signals from the at least two color channels (R, G, B).

8. The imaging method as claimed in claim 1, further comprising using an automated algorithm to separate the fluorescence from the excitation light.

9. The imaging method as claimed in claim 1, further comprising converting signals of the color channels (R, G, B) into a color space that has a saturation value as a coordinate or a degree of freedom.

10. The imaging method as claimed in claim 9, wherein the color space is an HSV color space, and color saturation values that are obtained from the signals by the conversion are assigned to corresponding components of the fluorescence or of the excitation light.

11. The imaging method as claimed in claim 1, further comprising producing an image signal which corresponds to an intensity distribution of the fluorescence or of the excitation light.

12. The imaging method as claimed in claim 1, further comprising storing a color vector in each case as a unit vector for at least one of an overall intensity detected by the color channels (R, G, B), the light source (4), or the fluorophore (5), and the component of the fluorescence of the excitation light is established by computational projection of a detected intensity vector along the color vector of the light source and of the fluorophore onto the color vector of the fluorophore and of the light source.

13. The imaging method as claimed in claim 1, wherein the sensor (6) is an image sensor and is at least one of a Bayer sensor or has at least three color channels, and exactly one said sensor (6) is used for imaging purposes.

14. The imaging method as claimed in claim 1, wherein the sensor (6) has different spectral filters or pixels which are arranged in such a way that an entire spectral range to be detected is capturable by spatially adjacent one of the spectral filters or pixels.

15. The imaging method as claimed in claim 1, wherein the sensor (6) has sensor elements for detecting red, green and blue light, and the sensor elements are used to detect the fluorescence.

16. The imaging method as claimed in claim 1, wherein the second spectral range (2) of the fluorescence lies partly or completely above a wavelength of 780 nm or lies partly or completely below a wavelength of 700 nm.

17. The imaging method as claimed in claim 1, further comprising using a narrowband light source (4) to excite the fluorophore (5), wherein an emission wavelength of the light source (4), at which the fluorophore (5) exhibits maximum light emission, lies outside of the second spectral range (2) of the fluorescence or wherein an emission wavelength, at which the light source (4) exhibits maximum light emission, is shorter than an absorption wavelength of the fluorophore (5), at which the fluorophore (5) exhibits maximum light absorption.

18. The imaging method as claimed in claim 1, further comprising the sensor (6) detecting light in a third spectral range (3) besides, or in addition to, the excitation light and the fluorescence, and sensitivities of the at least two color channels are distributed differently in the third spectral range (3) and in the first or the second spectral range (1, 2).

19. The imaging method as claimed in claim 1, further comprising obtaining a first image with the sensor (6) by detecting the excitation light in the first spectral range (1) or by detecting the fluorescence in the second spectral range (2); and obtaining a second image with the sensor (6) by detecting broadband illumination light in a third spectral range (3), wherein the light source (4) for the excitation light is a narrowband first light source (4), and a second light source (7) is used to produce the illumination light.

20. The imaging method as claimed in claim 19, wherein detecting the fluorescence or excitation light and detecting the illumination light are undertaken alternately, and the two light sources (4, 7) are operated alternately.

21. An image recording apparatus (8), comprising: a sensor and a data processor configured to separate the fluorescence from the excitation light by irradiating a fluorophore (5) with excitation light from light source (4), detecting light emitted by the fluorophore (5) in a first spectral range (1) and a fluorescence emitted by the fluorophore (5) in a second spectral range (2) using the sensor (6), wherein the sensor (6) has at least two color channels (R, G, B), sensitivities of said at least two color channels are distributed differently in the first spectral range (1) and in the second spectral range (2), and the at least two color channels (R, G, B) in each case detect the excitation light in the first spectral range (1) and the fluorescence in the second spectral range (2).
